(19) United States
(12) Patent Application Publication
(10) Pub. No.: US 2020/0175340 A1
(43) Pub. Date: Jun. 4, 2020

(54) METHOD AND SYSTEM FOR EVALUATING QUALITY OF MEDICAL IMAGE DATASET FOR MACHINE LEARNING

(71) Applicant: AJOU UNIVERSITY INDUSTRY-ACADEMIC COOPERATION FOUNDATION, Suwon-si (KR)

(72) Inventors: Jung Won LEE, Seoul (KR); Ye Seul PARK, Incheon (KR); Dong Yeon YOO, Suwon-si (KR); Chang Nam LIM, Seoul (KR)

(73) Assignee: AJOU UNIVERSITY INDUSTRY-ACADEMIC COOPERATION FOUNDATION, Suwon-si (KR)

(21) Appl. No.: 16/655,443

(22) Filed: Oct. 17, 2019

(30) Foreign Application Priority Data
Nov. 30, 2018 (KR) 10-2018-0152863

(51) Int. Cl. G06K 9/62; G06N 20/00; G16H 30/40

(52) U.S. Cl. CPC G06K 9/6262; G06K 9/6259; G16H 30/40; G06N 20/00

(57) ABSTRACT

The present disclosure relates to a method for evaluating quality of a medical image dataset and a system thereof capable of confirming whether medical image data is suitable to be used for machine learning. Evaluation items may include data normality which means a ratio of normal frames in all frames; learning fitness which means a ratio of labeled or labelable frames in the received data; and anatomical completeness which means a ratio of anatomical elements included in the received data against anatomical elements based on medical standards.

### CROSS-REFERENCE TO RELATED APPLICATIONS

This application claims the priority of Korean Patent Application No. 10-2018-0152863 filed on Nov. 30, 2018, in the Korean Intellectual Property Office, the disclosure of which is incorporated herein by reference.

### BACKGROUND

### Field

The present disclosure relates to a method for evaluating quality of a medical image dataset for machine learning and a system thereof, and more particularly, to a method for evaluating quality of a dataset and a system thereof to confirm whether medical image data is suitable to be used for machine learning.

### Description of the Related Art

Google has collected a large amount of retinal fundus photographs to develop an algorithm for detecting diabetic retinopathy. However, since most of the collected medical image data could not be labeled, Google has performed a labeling operation with the help of medical experts and at this time, a tool capable of assisting the labeling operation has been developed.

The tool developed by Google provides a large help to select learnable data for a design of a diabetic retinopathy detection algorithm and in the related paper, a difference of the performance of the learned algorithms has been explained and verified according to the image quality and the label of the image.

However, a program or algorithm has not been developed to evaluate the quality of the collected data whether the collected data is sufficient currently to be applied to the machine learning. Therefore, even though the medical image data is actually collected from the medical institution, the collected data is not sufficient to be applied to the machine learning and thus the derived result is not good in many cases.

For example, the number of data in a normal group and the number of data in an abnormal group should be similar to each other, and the medical image data should be appropriately labeled so that it can be seen that the quality is excellent.

### SUMMARY

The present disclosure is contrived to solve the problems, and an object of the present disclosure is to provide a reference for evaluating the quality of a medical image dataset for machine learning, and to provide a method for evaluating the entire collected data and a system thereof.

The technical objects of the present disclosure are not restricted to the aforementioned technical objects, and other objects of the present disclosure, which are not mentioned above, will become more apparent to one of ordinary skill in the art to which the present disclosure pertains by referencing the detailed description of the present disclosure given below.

An exemplary embodiment of the present disclosure provides a method for evaluating quality of medical image data for machine learning including: receiving, by a requirement definition unit, requirements according to a machine learning purpose; receiving, by a data reception unit, medical image data for machine learning; and evaluating, by a data evaluation unit, evaluation items in which the requirement received by the requirement definition unit is applied to the medical image data for machine learning received by the data reception unit.

According to an exemplary embodiment of the present disclosure, the evaluation items may include data normality which means a ratio of normal frames in all frames; learning fitness which means a ratio of labeled or labelable frames in the received data; and anatomical completeness which means a ratio of anatomical elements included in the received data against anatomical elements based on medical standards.

The data normality may be calculated by the following Equation 1.

$$Data Normality = \frac{Nor}{T} \\ ( Nor = 1 - {\sum }_{i = 1}^{8} {N}_{i} ) \qquad Equation 1$$

(Here, T represents the total number of frames, Nor represents the number of normal frames, Nᵢ represents the number of i-th type obstacle frames, and i represents an index of the obstacle type.)

The learning fitness may be calculated by the following Equation 2.

$$Learning Fitness = \frac{L}{T} \qquad Equation 2$$

(Here, T means the total number of frames and L means the number of labeled or labelable frames.)

The anatomical completeness may be calculated by the following Equation 3.

$$Anatomical Completeness = α \times \frac{MF}{{T}_{MF}} + ( 1 - α ) \times \frac{OF}{{T}_{OF}} \qquad Equation 3$$

(Here, MF is the number of required features (Mandatory Features) found, T_{MF} is the total number of required features, OF is the number of optional features found, T_{OF} is the total number of optional features, and α means a ratio factor determined according to the learning purpose.)

In the receiving of the requirements according to the machine learning purpose, the requirement definition unit may determine obstacle data defined according to a machine learning purpose, and in the evaluating of the evaluation items, data from which the obstacle data is removed may be evaluated.

Another exemplary embodiment of the present disclosure provides a system for evaluating quality of medical image dataset for machine learning including: a requirement definition unit configured to receive requirements according to a machine learning purpose; a data reception unit configured to receive medical image data for machine learning; and a data evaluation unit configured to evaluate evaluation items in which the requirement received by the requirement definition unit is applied to the medical image data for machine learning received by the data reception unit, in which the data evaluation unit includes, as evaluation items, data normality which means a ratio of normal frames in all frames; learning fitness which means a ratio of labeled or labelable frames in the received data; and anatomical completeness which means a ratio of anatomical elements included in the received data against anatomical elements based on medical standards.

According to the exemplary embodiment of the present disclosure, it is possible to determine whether the collected data is suitable to perform the machine learning by evaluating a collected medical image dataset.

Further, it is possible to determine whether the data is suitable for the data according to a learning purpose by evaluating data with another reference according to a purpose to be learned.

According to the exemplary embodiment of the present disclosure, by evaluating whether the medical image data is suitable for learning, it is possible to provide a developer with a quality evaluation level for the collected data, thereby assisting more effective learning data collection and learning network design.

The effects of the present disclosure are not limited to the aforementioned effect, and other effects, which are not mentioned above, will be apparent to a person having ordinary skill in the art from the following disclosure.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a block diagram of a system for evaluating quality of a medical image dataset for machine learning according to an exemplary embodiment of the present disclosure;

FIG. 2A illustrates an image defined as a positive image according to a learning purpose, and FIG. 2B illustrates an image defined as a negative image according to a learning purpose;

FIG. 3 is a flowchart of a method for evaluating quality of a medical image dataset for machine learning according to an exemplary embodiment of the present disclosure;

FIG. 4 is a flowchart of a method for evaluating quality of a medical image dataset for machine learning according to another exemplary embodiment of the present disclosure;

FIG. 5 illustrates evaluation items for evaluating quality of a medical image dataset for machine learning according to an exemplary embodiment of the present disclosure; and

FIG. 6 illustrates a result for evaluating quality of data according to an exemplary embodiment of the present disclosure.

### DETAILED DESCRIPTION OF THE PREFERRED EMBODIMENT

The present disclosure may be variously modified and have various embodiments and specific exemplary embodiments will be described in detail with reference to drawings. However, this does not limit the present disclosure to specific exemplary embodiments, and it should be understood that the present disclosure covers all the modifications, equivalents and replacements included within the idea and technical scope of the present disclosure. In describing each drawing, reference numerals refer to like elements.

Terms including as first, second, A, B, and the like are used for describing various constituent elements, but the constituent elements are not limited by the terms. The terms are used only to discriminate one constituent element from another component. For example, a first component may be referred to as a second component, and similarly, the second component may be referred to as the first component without departing from the scope of the present disclosure. A term ‘and/or’ includes a combination of a plurality of associated disclosed items or any item of the plurality of associated disclosed items.

It should be understood that, when it is described that a component is “connected to” or “accesses” another component, the component may be directly connected to or access the other component or a third component may be present therebetween. In contrast, it should be understood that, when it is described that an element is “directly coupled” or “directly connected” to another element, it is understood that no element is not present between the element and the another element.

Terms used in the present application are used only to describe specific exemplary embodiments, and are not intended to limit the present disclosure. A singular form may include a plural form if there is no clearly opposite meaning in the context. In the present application, it should be understood that term “include” or “have” indicates that a feature, a number, a step, an operation, a component, a part or the combination thereof described in the specification is present, but does not exclude a possibility of presence or addition of one or more other features, numbers, steps, operations, components, parts or combinations thereof, in advance.

If it is not contrarily defined, all terms used herein including technological or scientific terms have the same meanings as those generally understood by a person with ordinary skill in the art. Terms which are defined in a generally used dictionary should be interpreted to have the same meaning as the meaning in the context of the related art, and are not interpreted as an ideal meaning or excessively formal meanings unless clearly defined in the present application.

Throughout the specification and claims, unless explicitly described to the contrary, a case where any part “includes” any component will be understood to imply the inclusion of stated components but not the exclusion of any other component.

Hereinafter, preferred exemplary embodiments of the present disclosure will be described in detail with reference to the accompanying drawings.

Throughout the specification, a capsule endoscopic image is described as an example of medical image data, but the present disclosure is not necessarily limited thereto.

On the other hand, throughout the specification, ‘obstacle’ may refer to data which is not suitable to be applied to machine learning even with medical image data with good resolution. For example, when there are too many bubbles or foreign substances in the capsule endoscope image, the image may be referred to as an obstacle image when the shape of the internal organs is difficult to be recognized. Throughout the specification, the obstacle image may also be referred to as ‘obstacle data’.

In addition, the medical image data refers to a dataset collected from a medical institution, and evaluating the quality of the dataset means evaluating whether the data is collected so that the collected degree is suitable for performing machine learning.

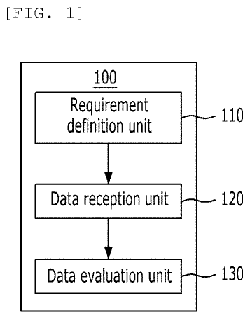

FIG. 1 is a block diagram of a system for evaluating quality of a medical image dataset for machine learning according to an exemplary embodiment of the present disclosure.

Referring to FIG. 1, a system 100 for evaluating quality of medical image data for machine learning according to an exemplary embodiment of the present disclosure may include a requirement definition unit 110 for receiving requirements according to a machine learning purpose; a data reception unit 120 for receiving medical image data for machine learning; and a data evaluation unit 130 for evaluating evaluation items in which the requirement received by the requirement definition unit is applied to the medical image data for machine learning received by the data reception unit.

The requirement definition unit 110 may receive definitions of the image from a user's input according to the learning purpose. That is, the requirement may mean a definition of an image.

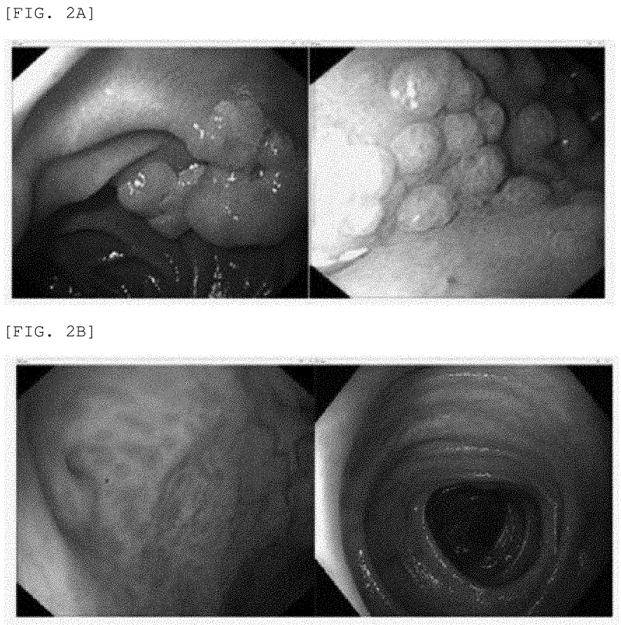

For example, when the purpose of learning about data is machine learning for detecting lesions, an image having lesions (polyps) as illustrated in FIG. 2A may be defined as a positive image, and an image having no lesions (polyps) as illustrated in FIG. 2B may be defined as a negative image.

Meanwhile, if the purpose of learning about the data is learning to track a location of a capsule, the anatomical structure of the gastrointestinal tract is labeled. Then, a positive image or a negative image is defined according to a network design of a developer. For example, if the location of the capsule is tracked through cross-point recognition of the gastrointestinal tract, an image with the Z-Line, the pyloric valve and ileocecal valve is defined as a positive image, and an image with the stomach, the esophagus and the small intestine is defined as a negative image.

The requirement definition unit 110 may also receive definitions of the obstacle data from the user. For example, an image with many bubbles or foreign substances that become an obstacle to the machine learning may be defined as an obstacle data image.

Meanwhile, the obstacle data may be defined differently depending on a machine learning purpose. For example, when the learning purpose is learning to track the location of the capsule endoscope, an image with too many or too little lesions may be defined as an obstacle data image in learning.

The data reception unit 120 may receive a capsule endoscopic image stored in a medical institution or a server of a hospital.

Whether the received medical image data is suitable to be used for the machine learning may be evaluated by the data evaluation unit 130.

The data evaluation unit 130 may evaluate quality of the received data by setting as evaluation items, data normality which means a ratio of normal frames in all frames; learning fitness which means a ratio of labeled or labelable frames in the received data; and anatomical completeness which means a ratio of anatomical elements included in the received data against anatomical elements based on medical standards.

More specifically, the data normality indicates a degree to which normal data except for the obstacle data is collected, and the learning fitness indicates a degree to which the data may be used for machine learning according to the degree of labeling of the collected data. The label means that the presence or absence of the polyp, an anatomical location, etc. are indicated on the medical image data.

Meanwhile, the anatomical completeness indicates a degree to which anatomical elements all are included in the collected data.

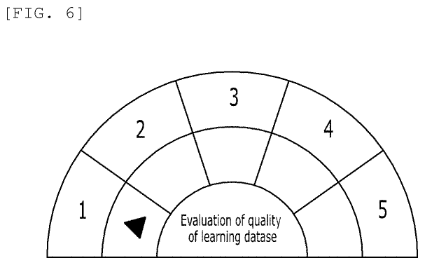

The data normality, the learning fitness, and the anatomical completeness constituting the evaluation items will be described below with reference to FIG. 5.

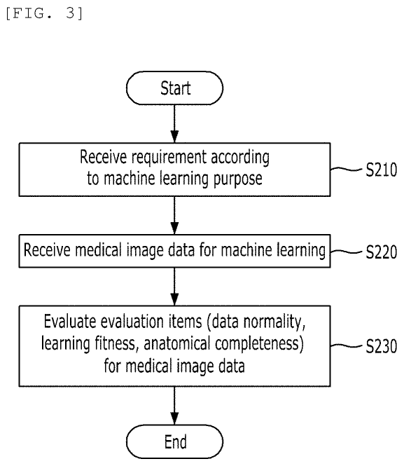

FIG. 3 is a flowchart of a method for evaluating quality of a medical image dataset for machine learning according to an exemplary embodiment of the present disclosure.

Referring to FIG. 3, a method for evaluating quality of medical image data for machine learning according to an exemplary embodiment of the present disclosure may include receiving, by a requirement definition unit, requirements according to a machine learning purpose (S210); receiving, by a data reception unit, medical image data for machine learning (S220); and evaluating, by a data evaluation unit, evaluation items in which the requirement received by the requirement definition unit is applied to the medical image data for machine learning received by the data reception unit (S230).

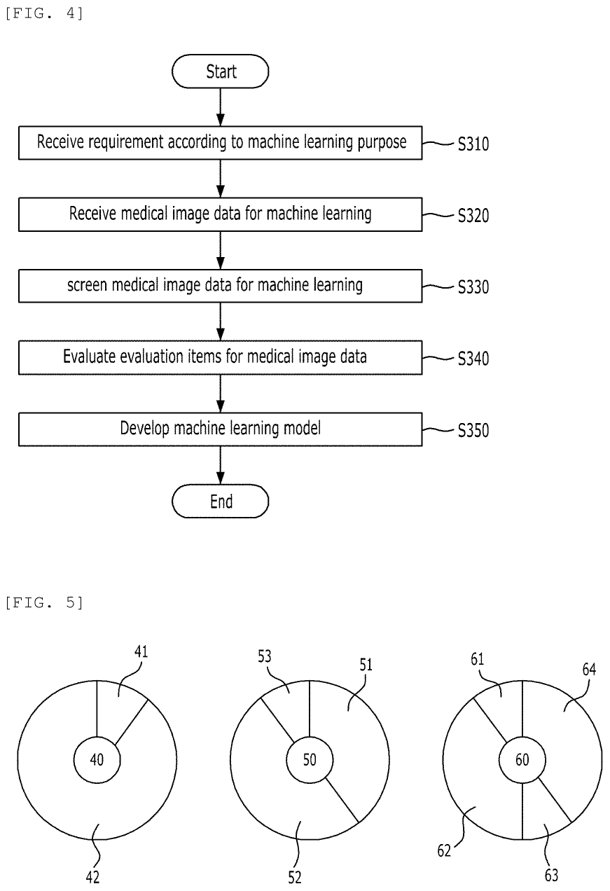

FIG. 4 is a flowchart of a method for evaluating quality of a medical image dataset for machine learning according to another exemplary embodiment of the present disclosure.

Referring to FIG. 4, receiving requirements according a machine learning purpose (S310), and receiving medical image data for machine learning (S320) are the same as those of FIG. 3, and screening medical image data for machine learning (S330) may be further included.

The screening of the medical image data (S330) is performing analysis of image frames with a learning purpose, analysis of obstacle image frames, and analysis of anatomical elements included in the image, as a step of confirming all medical image data sets by hospital staffs or artificial intelligence (AI) algorithms.

Meanwhile, a method of determining the obstacle image may use algorithms such as Gabor Filter, Gray Level Co-Occurrence Matrix (GLCM), Speeded Up Robust Features (SURF), Histogram, Wavelet Transform, and Convolution Neural Network (CNN) in the related art.

When the screening of the medical image data (S300) is performed, a label corresponding to the analyzed content may be included in a frame constituting the medical image data.

The method for evaluating the quality of the medical image data according to FIG. 4 may further include developing a machine learning model (S350) after evaluating the evaluation items for the medical image data (S340).

FIG. 5 illustrates evaluation items for evaluating quality of a medical image dataset for machine learning according to an exemplary embodiment of the present disclosure.

Data normality 40 is an evaluation item indicating the degree of collection of normal data, which may be calculated by the following Equation 1.

$$Data Normality = \frac{Nor}{T} \\ ( Nor = 1 - {\sum }_{i = 1}^{8} {N}_{i} ) \qquad Equation 1$$

Here, T represents the total number of frames, Nor represents the number 42 of normal frames, Nᵢ represents the number 41 of i-th type obstacle frames, and i represents an index of the obstacle type.

In the exemplary embodiment of the present disclosure, types of obstacle include 8 types of obstacle, such as a bubbled capsule endoscopic image (Bubble), an image of floating food residues in digesting (Residue), a fuzzy, dark or bright image (Fuzzy, Dark, Bright Image), an image with obstacle due to bad communication (Bad Communication), an image of the gastrointestinal tract filled with intestinal juices (Intestinal Juice), an image showing foreign substances (Foreign Body Ingestion), an adhesive closing image due to food bolus (Food Bolus Impaction), and an image with many lesions enough to block the structure of the gastrointestinal tract (Lesion Image), which may be defined as obstacle.

On the other hand, all the obstacle types are defined as obstacle and are not included in Equation 1. Depending on the purpose of machine learning, only some types of obstacle may be classified as obstacle data among the types of obstacle described above.

According to the exemplary embodiment of the present disclosure, grades of normality for each percentage of normality 40 may be divided as shown in Table 1 below.

TABLE 1

| Each evaluation item (%) | Grade |
|---|---|
| 90 to 100 | 5 |
| 80 to 90 | 4 |
| 70 to 80 | 3 |
| 60 to 70 | 2 |
| Less than 60 | 1 |

Learning fitness 50 is an evaluation item indicating a degree to which the data may be used for learning according to a labeling degreed of the collected data, which may be calculated by the following Equation 2.

$$Learning Fitness = \frac{L}{T} \qquad Equation 2$$

Here, T means the total number of frames and L means the number of labeled or labelable frames.

The frames of the received data may be labeled 51 or not labeled. Even if the frame not labeled, the developer can determine a label of frames in a nearby section as the labelable frames 52 based on the labeled frame.

More specifically, L means a sum of Frames Labeled the Positive Learning Purpose (LLP), Frames Labeled the Negative Learning Purpose (LLN), and Frames Possible to Label Learning Purpose (LUP) 52.

That is, L means the number obtained by subtracting Frames Impossible to Label Learning Purpose (LUI) 53 from the total number T of frames.

The learning fitness evaluation item may also be graded by percentage, as shown in Table 1.

The anatomical completeness 60 is an evaluation item capable of determining a degree in which the anatomical elements are included in the collected data.

The anatomical elements are based on the anatomical elements presented by minimal standard terminology for gastrointestinal endoscopy (MST) and capsule endoscopy structured terminology (CEST), which are medical standards for a capsule endoscope, and a ratio of anatomical elements confirmed in an image photographed by the capsule endoscope is calculated as an evaluation item.

That is, by anatomically analyzing the data collected through the anatomical completeness, it can be confirmed whether there is an anatomical element identified in the capsule endoscope. The anatomical elements may be divided into required features and optional features. The required features are anatomical features that may be found in everyone, and the optional features are features that may be found only in some patients.

The anatomical completeness 60 is calculated by Equation 3.

$$Anatomical Completeness = α \times \frac{MF}{{T}_{MF}} + ( 1 - α ) \times \frac{OF}{{T}_{OF}} \qquad Equation 3$$

Where MF is the number of required features found (63), T_{MF} is the total number of required features (63+64), OF is the number of optional features found (61), T_{OF} is the total number of optional features (61+62), and α means a ratio factor determined according to the learning purpose.

For example, the required features may include gastrointestinal landmarks and gastrointestinal tract junctions, and the optional features may include findings by cross-sectional location and findings by degree elevation of lesions.

In addition, scoring the anatomical completeness may be shown as Table 2 below.

TABLE 2

| Required discovery element completeness (%) $α \times \frac{MF}{{T}_{MF}}$ | Score | Optional discovery element completeness (%) $( 1 - α ) \times \frac{OF}{{T}_{OF}}$ | Score |
|---|---|---|---|
| (90-100)% × α | 5 | (90-100)% × (1 − α) | 5 |
| (80-90)% × α | 4 | (80-90)% × (1 − α) | 4 |
| (70-80)% × α | 3 | (70-80)% × (1 − α) | 3 |
| (60-70)% × α | 2 | (60-70)% × (1 − α) | 2 |
| Less than 60% × α | 1 | Less than 60% × (1 − α) | 1 |

FIG. 6 illustrates a result for evaluating quality of data according to an exemplary embodiment of the present disclosure. The total quality grade may be represented as illustrated in FIG. 6 by combining grades or scores of data normality, learning fitness, and anatomical completeness as shown in Tables 1 and 2 above.

In the exemplary embodiment of the present disclosure, the higher the grade, the higher the quality of the data, but the exemplary embodiment is not necessarily limited thereto.

According to the exemplary embodiment of the present disclosure, a case of learning a location of the capsule endoscope based on the gastrointestinal cross point will be described as an example.

A type of label includes gastrointestinal junction (GI Junction), which is a positive image, and gastrointestinal landmark (GI Landmark), which is a negative image.

The GI junction includes a Z-line, the pyloric valve, the ileocecal valve, and the GI landmark includes the esophagus, the stomach, the small intestine, and the large intestine.

Medical image data frames collected from 12 patients were used and a total collection time is 6 days 4 hours 5 minutes seconds. The duplicated frames are removed from the collected medical image data frames, and a total of 253,003 frames are used for evaluation.

The number of labelable frames (L, 51+52) is 244,835, and the number of non-labelable frames (LUI, 53) is 8,168. Further, the number of frames 41 determined as obstacle data is 15,784.

The total number of required features (T_{MF}, 63+64) is 12, the number of required features found (MF, 63) is 8, the total number of optional features (T_{OF}, 61+62) is 29, and the number of optional features found (OF, 61) is 2.

Data normality according to an exemplary embodiment of the present disclosure is calculated as 94% by Equation 1 (T=253,003, N=15,784).

$$Data Normality = 1 - \frac{15784}{\text{253,003}} \times 100 = 94 \%$$

Learning Fitness according to an exemplary embodiment of the present disclosure is calculated as 96.7% by Equation 2 (T=253,003, L=244,835).

$$Learning Fitness = \frac{\text{244,835}}{\text{253,003}} \times 100 = 96.7 \%$$

Anatomical completeness according to an exemplary embodiment of the present disclosure is calculated as 66.6% by Equation 3 (α=1, MF=8, T_{MF}=12, OF=2, T_{OF}=29). In this case, since a learning purpose is for learning the location of the capsule endoscope, the ratio (1−α) for the optional features appearing according to the lesion may be determined as 0, and the ratio (α) for the required features may be determined as 1.

$$Anatomical Completeness = 1 \times \frac{8}{12} + 0 \times \frac{2}{29} = 66.6 \%$$

When all of the evaluation items are described, it can be seen that the data normality, the learning fitness, and the anatomical completeness are relatively good, so that the developer can determine that collected dataset are suitable for learning the location of the capsule endoscope based on the cross-section of the gastrointestinal tract.

According to another exemplary embodiment of the present disclosure, a case for learning detection of bleeding lesions will be described as an example.

A type of label is divided into bleeding as a positive image and normal as a negative image.

Medical image data frames collected from 12 patients were used and a total collection time is 6 days 4 hours 5 minutes seconds. The duplicated frames are removed from the collected medical image data frames, and a total of 253,003 frames are used for evaluation.

The number of labelable frames (L, 51+52) is a total of 2737 including 3 bleeding image frames and 2734 normal image frames, and the number of non-labelable frames (LUI, 53) is 250,266. Further, the number of frames 41 determined as obstacle data is 15,784.

The total number of required features (T_{MF}, 63+64) is 12, the number of required features found (MF, 63) is 8, the total number of optional features (T_{OF}, 61+62) is 29, and the number of optional features found (OF, 61) is 2.

Data normality according to an exemplary embodiment of the present disclosure is calculated as 94% by Equation 1 (T=253,003, N=15,784).

$$Data Normality = 1 - \frac{15784}{\text{253,003}} \times 100 = 94 \%$$

Learning Fitness according to an exemplary embodiment of the present disclosure is calculated as 1.1% by Equation 2 (T=253,003, L=2,737).

$$Learning Fitness = \frac{\text{2,737}}{\text{253,003}} \times 100 = 1.1 \%$$

Anatomical completeness according to an exemplary embodiment of the present disclosure is calculated as 24% by Equation 3 (α=0.3, MF=8, T_{MF}=12, OF=2, T_{OF}=29). In this case, since a learning purpose is for detecting the bleeding lesion, a ratio (1−α) for the optional features being appeared whether the lesion occurs may be determined high. According to an exemplary embodiment of the present invention, the ratio may be determined as 0.7, and a ratio (α) for the required features may be determined as 0.3.

$$Anatomical Completeness = 0.3 \times \frac{8}{12} + 0.7 \times \frac{2}{29} = 24 \%$$

When describing all the evaluation items, the data normality is high and the learning fitness and the anatomical completeness are relatively much low, so that the developer may determine that the dataset is not suitable to be used for learning for detecting the bleeding lesion.

The above description just illustrates the technical spirit of the present disclosure and various changes and modifications can be made by those skilled in the art to which the present disclosure pertains without departing from a required characteristic of the present disclosure. Accordingly, the various embodiments disclosed in the present disclosure are not intended to limit the technical spirit but describe the present disclosure and the technical spirit of the present disclosure is not limited by the following embodiments. The protective scope of the present disclosure should be construed based on the appended claims, and all the technical spirits in the equivalent scope thereof should be construed as falling within the scope of the present disclosure.

What is claimed is:

1. A method for evaluating quality of a medical image dataset for machine learning using a system including a requirement definition unit, a data reception unit, and a data evaluation unit, the method comprising:
receiving, by the requirement definition unit, requirements according to a machine learning purpose;
receiving, by the data reception unit, medical image data for machine learning; and
evaluating, by the data evaluation unit, evaluation items in which the requirement received by the requirement definition unit is applied to the medical image data for machine learning received by the data reception unit,
wherein the evaluation items include
data normality which means a ratio of normal frames in all frames;
learning fitness which means a ratio of labeled or labelable frames in the received data; and
anatomical completeness which means a ratio of anatomical elements included in the received data against anatomical elements based on medical standards.

2. The method of claim 1, wherein the data normality is calculated by the following Equation 1.
$$Data Normality = \frac{Nor}{T} \\ ( Nor = 1 - {\sum }_{i = 1}^{8} {N}_{i} ) \qquad Equation 1$$
(Here, T represents the total number of frames, Nor represents the number of normal frames, Nᵢ represents the number of i-th type obstacle frames, and i represents an index of the obstacle type.)

3. The method of claim 1, wherein the learning fitness is calculated by the following Equation 2.
$$Learning Fitness = \frac{L}{T} \qquad Equation 2$$
(Here, T means the total number of frames and L means the number of labeled or labelable frames.)

4. The method of claim 1, wherein the anatomical completeness is calculated by the following Equation 3.
$$Anatomical Completeness = α \times \frac{MF}{{T}_{MF}} + ( 1 - α ) \times \frac{OF}{{T}_{OF}} \qquad Equation 3$$
(Here, MF is the number of required features found, T_{MF} is the total number of required features, OF is the number of optional features found, T_{OF} is the total number of optional features, and α means a ratio factor determined according to the learning purpose.)

5. The method of claim 1, wherein in the receiving of the requirements according to the machine learning purpose, the requirement definition unit determines obstacle data defined according to a machine learning purpose, and
in the evaluating of the evaluation items, data from which the obstacle data is removed is evaluated.

6. A system for evaluating quality of a medical image dataset for machine learning, the system comprising:
a requirement definition unit configured to receive requirements according to a machine learning purpose;
a data reception unit configured to receive medical image data for machine learning; and
a data evaluation unit configured to evaluate evaluation items in which the requirement received by the requirement definition unit is applied to the medical image data for machine learning received by the data reception unit,
wherein the data evaluation unit includes, as evaluation items,
data normality which means a ratio of normal frames in all frames;
learning fitness which means a ratio of labeled or labelable frames in the received data; and
anatomical completeness which means a ratio of anatomical elements included in the received data against anatomical elements based on medical standards.

7. The system of claim 6, wherein the data normality is calculated by the following Equation 1.
$$Data Normality = \frac{Nor}{T} \\ ( Nor = 1 - {\sum }_{i = 1}^{8} {N}_{i} ) \qquad Equation 1$$
(Here, T represents the total number of frames, Nor represents the number of normal frames, Nᵢ represents the number of i-th type obstacle frames, and i represents an index of the obstacle type.)

8. The system of claim 6, wherein the learning fitness is calculated by the following Equation 2.
$$Learning Fitness = \frac{L}{T} \qquad Equation 2$$
(Here, T means the total number of frames and L means the number of labeled or labelable frames.)

9. The system of claim 6, wherein the anatomical completeness is calculated by the following Equation 3.
$$Anatomical Completeness = α \times \frac{MF}{{T}_{MF}} + ( 1 - α ) \times \frac{OF}{{T}_{OF}} \qquad Equation 3$$
(Here, MF is the number of required features found, T_{MF} is the total number of required features, OF is the number of optional features found, T_{OF} is the total number of optional features, ands means a ratio factor determined according to the learning purpose.)

10. The system of claim 6, wherein the requirement definition unit determines obstacle data defined according to a machine learning purpose, and
the data evaluation unit evaluates data from which the obstacle data is removed.
